(19) United States
(12) Patent Application Publication
(10) Pub. No.: US 2025/0041559 A1
(43) Pub. Date: Feb. 6, 2025

(54) Catheter Wrap Device

(71) Applicants: Michael Slatton, Palm Harbor, FL (US); David Couture, Palm Harbor, FL (US)

(72) Inventors: Michael Slatton, Palm Harbor, FL (US); David Couture, Palm Harbor, FL (US)

(21) Appl. No.: 18/792,126

(22) Filed: Aug. 1, 2024

Related U.S. Application Data
(60) Provisional application No. 63/516,946, filed on Aug. 1, 2023.

(51) Int. Cl. A61M 25/00

(52) U.S. Cl. CPC A61M 25/0017; A61M 25/0054; A61M 2205/02

(57) ABSTRACT

A catheter wrap device is provided. The device is comprised of a catheter wrap device that can be secured around a penis. The body of the device is comprised of a leakproof exterior surface and an interior surface comprised of an absorbent layer. The absorbent layer absorbs any blood, urine, and/or bodily fluids that may leak from the catheter tube and/or the user's penis. The leakproof exterior layer further controls said fluids from leaking onto the garments of the user.

### CROSS-REFERENCE TO RELATED APPLICATION

The present application claims priority to, and the benefit of, U.S. Provisional Application No. 63/516,946, which was filed on Aug. 1, 2023, and is incorporated herein by reference in its entirety.

### FIELD OF THE INVENTION

The present invention relates generally to the field of male catheters. More specifically, the present invention relates to a catheter wrap device that can be secured around the penis and catheter tube of a user to control fluid from leaking from the penis and tube onto the clothing of the user. Accordingly, the present disclosure makes specific reference thereto. Nonetheless, it is to be appreciated that aspects of the present invention are also equally applicable to other like applications, devices, and methods of manufacture.

### BACKGROUND

Some men are required to wear a catheter. This may be due to medical conditions or post-surgical needs. Said men invariably face challenges associated with leakage from the catheter. This leakage normally occurs at the insertion point of the catheter. Leakage of urine, blood, etc., may dampen and stain clothing. In addition, it can also put the individual in socially awkward and potentially embarrassing situations.

The current solution to control catheter leakage involves the use of adult diapers. To bolster the efficacy of this measure, many also wrap a feminine napkin or pad around the catheter tube right where it is inserted into the penis of the individual. However, this solution is far from ideal. Not only is it uncomfortable and cumbersome, causing a sense of bulkiness, but it's also not the most discrete option. As a result, the individual may become self-conscious.

Therefore, there exists a long-felt need in the art for a device that can be used to control leakage from a catheter. There also exists a long-felt need in the art for a catheter wrap device that can be used to control leakage from a catheter. More specifically, there exists a long-felt need in the art for a catheter wrap device that can be used to control leakage from a catheter at the insertion point of the catheter around a user's penis. In addition, there exists a long-felt need in the art for a catheter wrap device that can be used to control leakage from a catheter at the insertion point of the catheter around a user's penis wherein the device is not bulky. Further, there exists a long-felt need in the art for a catheter wrap device that can be used to control leakage from a catheter at the insertion point of the catheter around a user's penis wherein the device does not require the use of an adult diaper or feminine napkin.

The subject matter disclosed and claimed herein, in one embodiment thereof, comprises a catheter wrap device. The device is comprised of a catheter wrap device. The device can be secured around a penis and catheter tube of a user via a tab with an adhesive fastener and/or tape. The body of the device is comprised of a leakproof exterior surface and an interior surface comprised of an absorbent layer. The absorbent layer absorbs any blood, urine, and/or bodily fluids that may leak from the catheter tube and/or the user's penis with insertion point of the catheter tube. The leakproof exterior layer further controls said fluids from leaking onto the garments of the user.

In this manner, the catheter wrap device of the present invention accomplishes all the foregoing objectives and provides a device that can be used to control leakage from a catheter. More specifically, the device can be used to control leakage from a catheter at the insertion point of the catheter around a user's penis. While doing so, the device is not bulky and does not require the use of an adult diaper or feminine napkin.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a catheter wrap device. The device is comprised of a body with a leakproof exterior surface and an absorbent interior surface that can be secured around a penis and catheter tube. As a result, leakage from the catheter tube is absorbed and cannot penetrate the device in order to dampen/stain the user's clothes.

The body may be any shape in different embodiments to allow the device to be easily applied around the shaft and/or head of a user's penis. The interior surface of the body is comprised of at least one absorbent layer to collect any blood, urine, or other bodily fluids that exit the penis and/or catheter tube. The layer is also preferably made from an odor-absorbent material. Further, the exterior surface of the body is preferably made from a leakproof material to control any blood, urine, or other bodily fluids from seeping through the absorbent layer and onto the undergarments and/or clothing of the user.

To secure the body around the shaft and/or head of the user's penis, the body may be comprised of at least one tab having at least one peelable adhesive fastener. In a different embodiment, the body can be attached to itself using tape or any other medical-grade fastener of the like. In one embodiment, the entire device is disposable. In another embodiment, the absorbent material removably attaches (preferably via adhesive) to the interior surface. In this manner, the body can be washed and reused and wherein the absorbent layer can be replaced as needed.

The present invention is also comprised of a method of using the device. First, a device is provided comprised of a body comprised of a leakproof exterior surface and an interior surface comprised of an absorbent layer. Then, the body can be secured around the head, shaft, and/or head and shaft of a user's penis and around a catheter tube exiting the user's penis using at least one fastener.

In a second method of using the device, a device provided comprised of a body comprised of a leakproof exterior surface and an interior surface comprised of an absorbent layer. Then, the body can be secured around the head, shaft, and/or head and shaft of a user's penis and around a catheter tube exiting the user's penis using at least one fastener. Then, the absorbent material can be removed and replaced with new absorbent material. The body can be re-secured around the head, shaft, and/or head and shaft of a user's penis and around a catheter tube exiting the user's penis using at least one fastener.

Accordingly, the catheter wrap device of the present invention is particularly advantageous as it provides a device that can be used to control leakage from a catheter. More specifically, the device can be used to control leakage from a catheter at the insertion point of the catheter around a user's penis. While doing so, the device is not bulky and does not require the use of an adult diaper or feminine napkin. In this manner, the catheter wrap device overcomes the limitations of existing methods and devices used to control leakage from a male catheter that are known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a catheter wrap device of the present invention while unwrapped and positioned around a penis in accordance with the disclosed architecture;

FIG. 2 illustrates a perspective view of one potential embodiment of a catheter wrap device of the present invention while wrapped around a penis in accordance with the disclosed architecture;

FIG. 3 illustrates a flowchart of a method of using one potential embodiment of a catheter wrap device of the present invention in accordance with the disclosed architecture; and

FIG. 4 illustrates a flowchart of a method of using one potential embodiment of a catheter wrap device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a device that can be used to control leakage from a catheter. There also exists a long-felt need in the art for a catheter wrap device that can be used to control leakage from a catheter. More specifically, there exists a long-felt need in the art for a catheter wrap device that can be used to control leakage from a catheter at the insertion point of the catheter around a user's penis. In addition, there exists a long-felt need in the art for a catheter wrap device that can be used to control leakage from a catheter at the insertion point of the catheter around a user's penis wherein the device is not bulky. Further, there exists a long-felt need in the art for a catheter wrap device that can be used to control leakage from a catheter at the insertion point of the catheter around a user's penis wherein the device does not require the use of an adult diaper or feminine napkin.

The present invention, in one exemplary embodiment, is comprised of a catheter wrap device comprised of a body with a leakproof exterior surface and an absorbent interior surface that can be secured around a penis and catheter tube. As a result, leakage from the catheter tube is absorbed and cannot penetrate the device in order to dampen/stain the user's clothes.

The body may be any shape in different embodiments to allow the device to be easily applied around the shaft and/or head of a user's penis. The interior surface of the body is comprised of at least one absorbent layer. Said layer collects any blood, urine, or other bodily fluids that exit the penis and/or catheter tube. The layer is also preferably made from an odor-absorbent material. Further, the exterior surface of the body is preferably made from a leakproof material to control any blood, urine, or other bodily fluids from seeping through the absorbent layer and onto the undergarments and/or clothing of the user.

To secure the body around the shaft and/or head of the user's penis, the body may be comprised of at least one tab having at least one peelable adhesive fastener. In a different embodiment, the body can be attached to itself using tape or any other medical-grade fastener of the like. In one embodiment, the entire device is disposable. In another embodiment, the absorbent material removably attaches (preferably via adhesive) to the interior surface. In this manner, the body can be washed and reused and wherein the absorbent layer can be replaced as needed.

The present invention is also comprised of a method of using the device. First, a device is provided comprised of a body comprised of a leakproof exterior surface and an interior surface comprised of an absorbent layer. Then, the body can be secured around the head, shaft, and/or head and shaft of a user's penis and around a catheter tube exiting the user's penis using at least one fastener.

In a second method of using the device, a device provided comprised of a body comprised of a leakproof exterior surface and an interior surface comprised of an absorbent layer. Then, the body can be secured around the head, shaft, and/or head and shaft of a user's penis and around a catheter tube exiting the user's penis using at least one fastener. Then, the absorbent material can be removed and replaced with new absorbent material. The body can be re-secured around the head, shaft, and/or head and shaft of a user's penis and around a catheter tube exiting the user's penis using at least one fastener.

Accordingly, the catheter wrap device of the present invention is particularly advantageous as it provides a device that can be used to control leakage from a catheter. More specifically, the device can be used to control leakage from a catheter at the insertion point of the catheter around a user's penis. While doing so, the device is not bulky and does not require the use of an adult diaper or feminine napkin. In this manner, the catheter wrap device overcomes the limitations of existing methods and devices used to control leakage from a male catheter that are known in the art.

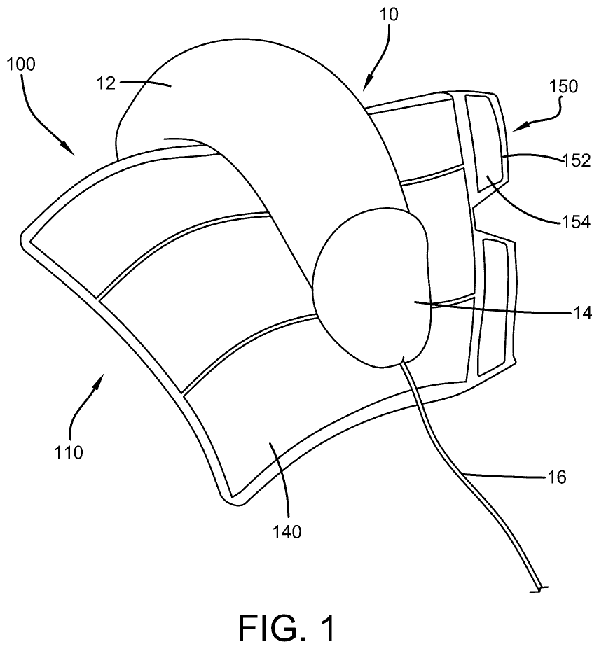

Referring initially to the drawings, FIG. 1 illustrates a perspective view of one potential embodiment of a catheter wrap device 100 of the present invention while unwrapped and positioned around a penis 100 in accordance with the disclosed architecture. The device 100 is comprised of a body 110 that can be secured around a penis 10 and a catheter tube 16. The body 110 may be comprised of a leakproof exterior surface 120 and an absorbent interior surface 130. As a result, leakage from the catheter tube 16 is absorbed and cannot penetrate the device 100 in order to dampen/stain the user's clothes.

The device 100 is comprised of a body 110. The body 110 may be any shape in different embodiments to allow the device 100 to be easily applied around the shaft and/or head 14 of a user's penis 10. In the preferred embodiment, the body 110 is generally trapezoidal in shape.

The interior surface 130 of the body 110 is comprised of at least one absorbent layer 140. The layer 140 may be comprised of an absorbent material such as, but not limited to, silica gel, cotton, charcoal, plant cellulose, hemp, bamboo fiber, etc., or any combination. The layer 140 absorbs any blood, urine, or other bodily fluids that exit the penis 10 and/or catheter tube 16. The layer 140 is also preferably made from an odor-absorbent material. In one embodiment, the absorbent layer 140 may not cover the entire interior surface 130. In another embodiment, the absorbent layer 140 may cover the entire interior surface 130 but may vary in absorbency at various points along the interior surface 130.

Further, the exterior surface 120 of the body 110 is preferably made from a leakproof material. Said material may be, but is not limited to, a leakproof laminated fabric and/or silicone. The leakproof material controls any blood, urine, or other bodily fluids from seeping through the absorbent layer 140 and onto the undergarments and/or clothing of the user.

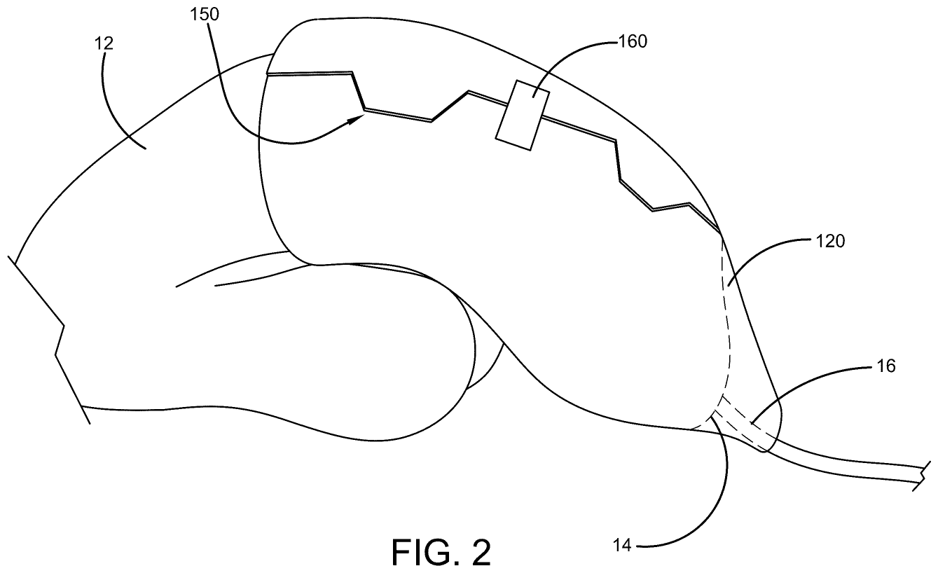

To secure the body 110 around the shaft 12 and/or head 14 of the user's penis 10, the body 110 may be comprised of at least one tab 150. The bottom surface 152 of the tab 150 may be comprised of at least one peelable adhesive fastener 154 (or any other fastener type). The fastener 154 can attach the bottom surface 152 of the tab 150 to the exterior surface 120 to secure the body 110 around the penis 10. The body 110 is preferably secured around only the head 14 of the penis 10, but may be sized and shaped to also secure around the shaft 12 of the penis 10. In a different embodiment, the body 110 can be attached to itself using tape 160 or any other medical-grade fastener of the like, as seen in FIG. 2.

In one embodiment, the entire device 100 is disposable. In another embodiment, the absorbent material 140 removably attaches (preferably via adhesive) to the interior surface 130. In this manner, the body 110 can be washed and reused and wherein the absorbent 140 layer can be replaced as needed.

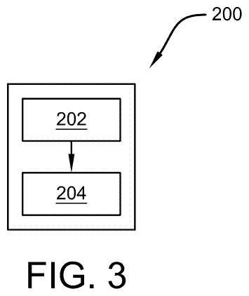

The present invention is also comprised of a method of using 200 the device 100, as seen in FIG. 3. First, a device 100 is provided comprised of a body 110 comprised of a leakproof exterior surface 120 and an interior surface 130 comprised of an absorbent layer 140 [Step 202]. Then, the body 110 can be secured around the head 14, shaft 12, and/or head 14 and shaft 12 of a user's penis 10 and around a catheter tube 16 exiting the user's penis 10 using at least one fastener 154, 160 [Step 204].

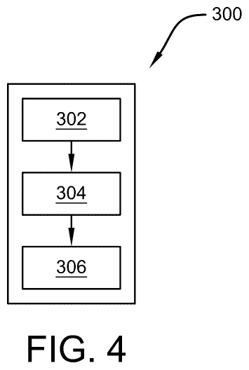

The present invention is also comprised of a method of using 300 the device 100, as seen in FIG. 4. First, a device 100 is provided comprised of a body 110 comprised of a leakproof exterior surface 120 and an interior surface 130 comprised of an absorbent layer 140 [Step 302]. Then, the body 110 can be secured around the head 14, shaft 12, and/or head 14 and shaft 12 of a user's penis 10 and around a catheter tube 16 exiting the user's penis 10 using at least one fastener 154, 160 [Step 304]. Then, the absorbent material 140 can be removed and replaced with new absorbent material. The body 110 can be re-secured around the head 14, shaft 12, and/or head 14 and shaft 12 of a user's penis 10 and around a catheter tube 16 exiting the user's penis 10 using at least one fastener 154, 160 [Step 306].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “catheter wrap device” and “device” are interchangeable and refer to the catheter wrap device 100 of the present invention.

Notwithstanding the foregoing, the catheter wrap device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the catheter wrap device 100 as shown in the FIGS. are for illustrative purposes only, and that many other sizes and shapes of the catheter wrap device 100 are well within the scope of the present disclosure. Although the dimensions of the catheter wrap device 100 are important design parameters for user convenience, the catheter wrap device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A catheter wrap device comprising:
a body comprised of an exterior surface and an interior surface;
an absorbent material attached to the interior surface; and
a fastener.

2. The catheter wrap device of claim 1, wherein the exterior surface is comprised of a leakproof material.

3. The catheter wrap device of claim 2, wherein the leakproof material is comprised of a laminated fabric material.

4. The catheter wrap device of claim 1, wherein the absorbent material is comprised of a silica gel, a cotton, a charcoal, a plant cellulose, a hemp, or a bamboo fiber material.

5. The catheter wrap device of claim 4, wherein the absorbent material is comprised of an odor-resistant material.

6. The catheter wrap device of claim 1, wherein the body is comprised of a trapezoidal shape.

7. The catheter wrap device of claim 1, wherein the fastener is comprised of a tape.

8. A catheter wrap device comprising:
a body comprised of an exterior surface, an interior surface, and a tab comprised of a fastener; and
an absorbent material removably attached to the interior surface.

9. The catheter wrap device of claim 8, wherein the exterior surface is comprised of a leakproof material.

10. The catheter wrap device of claim 9, wherein the leakproof material is comprised of a laminated fabric material.

11. The catheter wrap device of claim 8, wherein the absorbent material is comprised of a silica gel, a cotton, a charcoal, a plant cellulose, a hemp, or a bamboo fiber material.

12. The catheter wrap device of claim 11, wherein the absorbent material is comprised of an odor-resistant material.

13. The catheter wrap device of claim 8, wherein the body is comprised of a trapezoidal shape.

14. The catheter wrap device of claim 8, wherein the fastener is comprised of a peelable adhesive fastener.

15. A method of using a catheter wrap device, the method comprising the following steps:
providing a catheter wrap device comprised of a body comprised of a leakproof exterior surface and an interior surface comprised of an absorbent layer; and
securing the body around a penis and a catheter tubing exiting the penis via a fastener.

16. The method of using a catheter wrap device of claim 15, wherein the fastener is comprised of a tape.

17. The method of using a catheter wrap device of claim 15, wherein the fastener is comprised of a peelable adhesive located on a tab of the body.

18. The method of using a catheter wrap device of claim 15, wherein the body is secured around a head of the penis.

19. The method of using a catheter wrap device of claim 15, wherein the body is secured around a shaft of the penis.

20. The method of using a catheter wrap device of claim 15, wherein the body is secured around a head and a shaft of the penis.
